(19) United States
(12) Patent Application Publication
(10) Pub. No.: US 2025/0282699 A1
(43) Pub. Date: Sep. 11, 2025

(54) METHOD FOR PRODUCING 1,1,1,3,5,5,5-HEPTAFLUORO-2-PENTENE

(71) Applicant: KANTO DENKA KOGYO CO., LTD., Tokyo (JP)

(72) Inventor: Tomohiro TANIGUCHI, Okayama (JP)

(21) Appl. No.: 18/859,898

(22) PCT Filed: Apr. 27, 2023
(86) PCT No.: PCT/JP2023/016582

§ 371 Date: Oct. 24, 2024

(30) Foreign Application Priority Data
Apr. 28, 2022 (JP) 2022-074800

(51) Int. Cl. C07C 17/20; C07C 21/18

(52) U.S. Cl. CPC C07C 17/20; C07C 21/18

(57) ABSTRACT

An object of the present invention is to provide a method for producing a high-purity 1,1,1,3,5,5,5-heptafluoro-2-pentene (HFO-1447), particularly with a purity of more than 99%. Another object of the present invention is to provide a high-purity 1,1,1,3,5,5,5-heptafluoro-2-pentene (HFO-1447) and uses thereof. A method for producing 1,1,1,3,5,5,5-heptafluoro-2-pentene, including: (a) reacting 3-chloro-hexafluoro-2-pentene with hydrogen fluoride at a temperature of more than −10° C. and 20° C. or less in the presence of a metal halide catalyst to produce 3-chloro-1,1,1,3,5,5,5-heptafluoropentane; and (b) subjecting the 3-chloro-1,1,1,3,5,5,5-heptafluoropentane obtained in (a) to a dehydrochlorination reaction in the presence of an activated carbon catalyst to produce 1,1,1,3,5,5,5-heptafluoro-2-pentene.

### TECHNICAL FIELD

The present invention relates to a novel method for producing 1,1,1,3,5,5,5-heptafluoro-2-pentene (hereinafter sometimes referred to as HFO-1447), and particularly to a method for producing a high-purity 1,1,1,3,5,5,5-heptafluoro-2-pentene.

### BACKGROUND ART

Fluoroolefins are used as solvents, cleaning agents, foaming agents, and intermediates for functional materials, for example, and various production methods thereof have been proposed. In particular, 1,1,1,3,5,5,5-heptafluoro-2-pentene is useful as a synthetic intermediate because it has, in its molecule, a C═C double bond and fluorine substituents which serve as reaction initiation points. In addition, 1,1,1,3,5,5,5-heptafluoro-2-pentene has an ozone depletion potential of 0 due to the absence of chlorine atoms in its molecule: even when released into the natural world, it decomposes into low-molecular compounds due to the presence of a C═C double bond in its molecule; and it is noncombustible due to the presence of seven fluorine atoms in its molecule; and it is liquid at ordinary temperatures and pressures. Therefore, it is also useful as a foaming agent. For example, in Example 5 of PTL 1,1,1,1,3,3,5,5,5-octafluoropentane (hereinafter sometimes referred to as “HFC-458”) as a starting material is subjected to defluorination reaction with high surface AlF₃ as a catalyst at a temperature of 330° C. to produce HFO-1447, as shown in the following reaction equation. PTL 1 also discloses in Example 6 that HFO-1447 was used in a foaming agent composition, and in Example 7 that a mixed solvent containing HFO-1447 as a main component was used as a drying agent. However, as a result of confirmatory studies of the description in PTL 1, the present inventors have found the following: bis(trifluoromethyl) allene (hereinafter sometimes simply referred to as “allene compound”), which is not described in PTL 1, is generated as a by-product and is difficult to separate from (E)-HFO-1447 (boiling point: 52° C.); unreacted HFC-458 is difficult to separate from (Z)-HFO-1447 (boiling point: 78° C.); and as a result, when attempting to produce a high-purity HFO-1447, the yield is low. In addition, according to the studies by the present inventors, it has been found that the (E)-HFO-1447 containing an allene compound and the (Z)-HFO-1447 containing HFC-458 each are likely to deteriorate resins as compared to high-purity (E)-HFO-1447 and (Z)-HFO-1447.

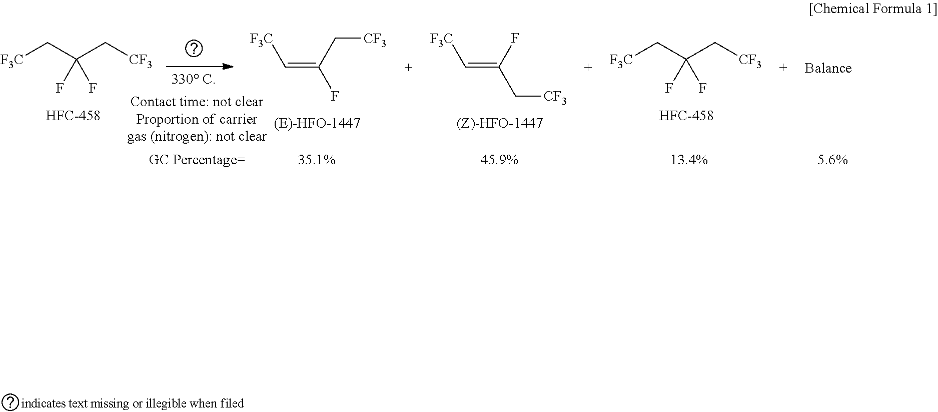

### CITATION LIST

### Patent Literature

PTL 1: Japanese Translation of PCT International Application Publication No. 2012-508778

### SUMMARY OF INVENTION

### Technical Problem

An object of the present invention is to provide a novel method for producing a 1,1,1,3,5,5,5-heptafluoro-2-pentene (hereinafter sometimes referred to as “HFO-1447”), and particularly a method for producing a high-purity HFO-1447 (particularly with a purity of more than 99%). Another object of the present invention is to provide a high-purity HFO-1447 and uses thereof.

### Solution to Problem

The present invention provides the following.

[1] A method for producing 1,1,1,3,5,5,5-heptafluoro-2-pentene, comprising:
(a) reacting 3-chloro-hexafluoro-2-pentene with hydrogen fluoride at a temperature of more than −10° C. and 20° C. or less in the presence of a metal halide catalyst to produce 3-chloro-1,1,1,3,5,5,5-heptafluoropentane; and
(b) subjecting the 3-chloro-1,1,1,3,5,5,5-heptafluoropentane obtained in (a) to a dehydrochlorination reaction in the presence of an activated carbon catalyst to produce 1,1,1,3,5,5,5-heptafluoro-2-pentene.

[2] The method according to [1], wherein the metal halide catalyst is selected from an antimony halide catalyst, a tin halide catalyst, a titanium halide catalyst, a niobium halide catalyst, a tantalum halide catalyst or a combination thereof.

[3] The method according to [1], wherein the metal halide catalyst is selected from antimony trichloride, antimony pentachloride, antimony trifluoride, antimony pentafluoride, tin tetrachloride, titanium tetrachloride, niobium pentafluoride, tantalum pentafluoride or a combination thereof.

[4] The method according to [1], wherein hydrogen fluoride is used at a molar equivalent ratio of 1 to 1.5 of hydrogen fluoride to 3-chloro-hexafluoro-2-pentene.

[5] The method according to [1], wherein the amount of the metal halide catalyst is 2 to 3 mol % based on the amount of 3-chloro-hexafluoro-2-pentene.

[6] The method according to [1] wherein the reacting in step (a) is carried out at a temperature of −5° C. to 10° C.

[7] The method according to [1], wherein the metal halide catalyst is selected from antimony trichloride, antimony pentachloride, antimony trifluoride, antimony pentafluoride, tin tetrachloride, titanium tetrachloride, niobium pentafluoride, tantalum pentafluoride or a combination thereof: hydrogen fluoride is used at a molar equivalent ratio of 1 to 1.5 of hydrogen fluoride to 3-chloro-hexafluoro-2-pentene; the amount of the metal halide catalyst is 2 to 3 mol % based on the amount of 3-chloro-hexafluoro-2-pentene; and the reacting in step (a) is carried out at a temperature of −5° C. to 10° C.

[8] A high-purity 1,1,1,3,5,5,5-heptafluoro-2-pentene obtained by the method according to any of [1] to [7].

[9] The high-purity 1,1,1,3,5,5,5-heptafluoro-2-pentene according to [8], having a purity of more than 99%.

### Advantageous Effects of Invention

The present inventors have changed the production process of HFO-1447, and as a result, have found a method for efficiently obtaining a high-purity HFO-1447. According to the present invention, neither production of or contamination with allene compounds or HFC-458 occurs, and thus, HFO-1447 with a high purity, particularly with a purity of more than 99%, can be efficiently produced. When HFO-1447 produced by other production methods and therefore containing impurities is used, deterioration of resins occurs. On the other hand, when the high-purity HFO-1447 of the present invention is used, deterioration of resins is suppressed and the range of applicable resins is expanded.

### BRIEF DESCRIPTION OF DRAWINGS

FIG. 1 is a schematic view of a reaction system used for the dehydrochlorination reaction in step (b).

### DESCRIPTION OF EMBODIMENTS

### [Action]

According to the present invention, a high-purity 1,1,1,3,5,5,5-heptafluoro-2-pentene (HFO-1447), particularly with a purity of more than 99% can be efficiently produced. According to the studies by the present inventors, the HFO-1447 obtained by the method proposed in the prior art literature (PTL 1) contains a starting material (HFC-458) and a product (an allene compound), which are difficult to separate from HFO-1447 by purification, and a high-purity HFO-1447 cannot be thus efficiently obtained. The present inventors have found that the HFO-1447 containing no allene compound can be obtained by (a) reacting 3-chloro-1,1,1,5,5,5-hexafluoro-2-pentene (hereinafter sometimes referred to as “HCFO-1446”), which is easily available and easily separated from a product, as a starting material with hydrogen fluoride at a temperature of more than −10° C. and 20° C. or less in the presence of a metal halide catalyst to produce 3-chloro-1,1,1,3,5,5,5-heptafluoropentane (hereinafter sometimes referred to as “HCFC-457”); and (b) subjecting the resulting 3-chloro-1,1,1,3,5,5,5-heptafluoropentane to a dehydrochlorination reaction in the presence of an activated carbon catalyst to produce HFO-1447. The HFO-1447 obtained by the method of the present invention does not contain any starting material or product that are difficult to separate, and therefore, the method of the present invention can efficiently produce HFO-1447 with a high purity that cannot be achieved by the prior art. Therefore, the high-purity HFO-1447 of the present invention is itself a novel invention.

According to the studies by the present inventors, it has also been found that the (E)-HFO-1447 containing an allene compound and the (Z)-HFO-1447 containing HFC-458 each are likely to deteriorate resins. Since the high-purity (E)-HFO-1447 and (Z)-HFO-1447 obtained by the method of the present invention do not contain the allene compound, which is contained in the HFO-1447 obtained by the method of the prior art, they are useful in all of the applications for which deterioration of resins is a problem.

[Step (a)]

In step (a), 3-chloro-hexafluoro-2-pentene is reacted with hydrogen fluoride at a temperature of more than −10° C. and 20° C. or less in the presence of a metal halide catalyst to produce 3-chloro-1,1,1,3,5,5,5-heptafluoropentane. Step (a) is characterized by using a specific starting material and subjecting it to a fluorination reaction within a specific temperature range in the presence of a specific catalyst. In step (a), only one molecule of hydrogen fluoride (HF) is added to one molecule of the starting material, and the target product can be therefore selectively obtained with almost no by-products.

3-chloro-hexafluoro-2-pentene, which is a starting material for producing 3-chloro-1,1,1,3,5,5,5-heptafluoropentane, can be easily produced by any known method or easily obtained as a reagent.

Examples of the metal halide catalyst include an antimony halide catalyst, a tin halide catalyst, a titanium halide catalyst, a niobium halide catalyst and a tantalum halide catalyst, which can be used singly or in combination of two or more thereof. Examples of the antimony halide catalyst include antimony trichloride, antimony pentachloride, antimony trifluoride and antimony pentafluoride: examples of the tin halide catalyst include tin tetrachloride: examples of the titanium halide catalyst include titanium tetrachloride: examples of the niobium halide catalyst include niobium pentafluoride; and examples of the tantalum halide catalyst include tantalum pentafluoride; and these can be used singly or in combination of two or more thereof.

Hydrogen fluoride is used preferably at a molar equivalent ratio of 0.5 to 10, more preferably 0.5 to 3 and most preferably 1 to 1.5 of hydrogen fluoride to 3-chloro-hexafluoro-2-pentene.

The amount of the metal halide catalyst is preferably 0.1 to 10 mol %, more preferably 0.5 to 5 mol % and most preferably 2 to 3 mol %, based on 100 mol % of the amount of 3-chloro-hexafluoro-2-pentene.

The reaction temperature is more than −10° C. and 20° C. or less, more preferably-10° C. to 15° C. and most preferably −5° C. to 10° C.

For the liquid phase reaction, hydrogen fluoride can be liquefied and used as a solvent. Since hydrogen fluoride has a boiling point of about 20° C., the liquid phase reaction is carried out within the temperature range of step (a). Therefore, step (a) can be carried out without using a pressure-resistant vessel such as an autoclave as a reaction vessel as long as the temperature is adequately managed.

[Step (b)]

In step (b), the 3-chloro-1,1,1,3,5,5,5-heptafluoropentane obtained in step (a) is subjected to a dehydrochlorination reaction in the presence of an activated carbon catalyst to produce HFO-1447. Since only elimination of one molecule of hydrogen chloride occur in this dehydrochlorination, the product is a mixture of E/Z isomers of HFO-1447.

The activated carbon catalyst may be one conventionally used in the dehydrochlorination of a halogenated alkane. Examples thereof include coconut shell charcoals for gas purification, catalysts and catalyst supports (granular Shirasagi GX, SX, CX and XRC manufactured by Takeda Pharmaceutical Company Limited: PCB manufactured by Toyo Calgon Co., Ltd.; Yashi Coal manufactured by TAIHEI CHEMICAL INDUSTRIAL CO., LTD.; and KURARAY COAL GG and GC, which can be used singly or in combination of two or more thereof. Since activated carbon is also used as a support, the activated carbon catalyst itself can be in the desired shape without the need to support the activated carbon on another support. Examples of the shape of the support include powder, granule, spherical, pellet, cylindrical, and honeycomb shapes.

The contact time with the catalyst is usually 0.1 to 300 seconds, preferably 5 to 120 seconds and more preferably 10 to 60 seconds, but is not limited thereto, and the contact time may be changed as appropriate.

The reaction temperature is preferably 150° C. to 350° C., more preferably 200° C. to 300° C., and most preferably 220° C. to 250° C.

### [Carrier Gas]

In carrying out the present invention, a carrier gas is used for diluting a starting material gas, and drying a reactor, for example. In particular, by flowing the carrier gas, substances such as a starting material and a reaction product can be moved within the reaction system while adjusting the concentrations thereof. The carrier gas to be selected is a gas that does not react with such substances. Examples of the carrier gas include nitrogen and a noble gas (such as helium, neon or argon). When using a carrier gas, it is usually mixed with substances such as a starting material and a reaction product such that the proportion of the carrier gas is 0 to 99%, more preferably 0 to 75%, and most preferably 0 to 50%, based on the total amount of the flowing substances.

### [Reaction System]

Examples of the material for the reaction system include corrosion-resistant metals such as stainless steel, Inconel, Monel, Hastelloy and nickel. Among these, nickel is preferred in view of corrosion resistance.

Examples of the system for carrying out the dehydrochlorination reaction in step (b) include a cylindrical tube equipped with a heater for adjusting the reaction temperature, packed with a catalyst having various shapes and configured to allow a starting material gas to flow from one end of the tube to the other. For the direction of flowing the starting material gas, it is preferable to gradually flow the starting material gas uniformly from top to bottom, in a case where the cylindrical tube packed with the catalyst is configured to extend vertically, and the reason for this is because gravity can be used to flow the starting material gradually. In a case where the starting material gas is flowed from bottom to top of the cylindrical tube configured to extend vertically, it is preferable to place a catalyst in the form of pellet with a large particle diameter at the bottom of the cylindrical tube and the catalyst in the form of powder with a small particle diameter at the top of the cylindrical tube, in terms of reaction efficiency.

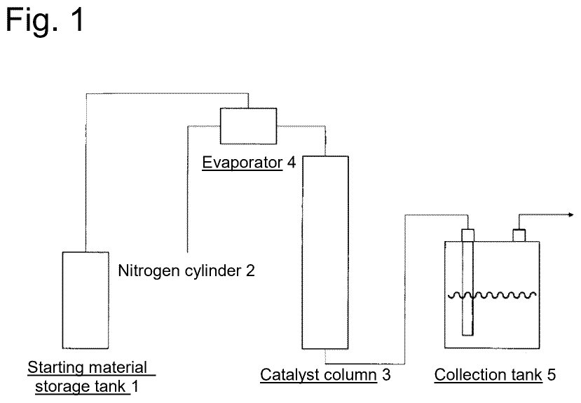

In FIG. 1, which schematically illustrates the specific example of the system for carrying out the dehydrochlorination reaction in step (b), this system is composed of starting material storage tank 1 for containing 3-chloro-1,1,1,3,5,5,5-heptachloropentane; nitrogen cylinder 2 for supplying nitrogen gas to evaporator 4: catalyst column 3 having a cylindrical shape, connected to the starting material storage tank by piping and set up vertically: evaporator 4 positioned immediately before the catalyst column; and collection tank 5 located downstream of the catalyst column. 3-Chloro-1,1,1,3,5,5,5-heptachloropentane (boiling point: 89° C.) in a liquid form is injected as it is by a syringe pump (not shown) into evaporator 4 heated to a temperature of its boiling point or more, and is vaporized in evaporator 4. The starting material gas is mixed with a carrier gas (nitrogen gas) in evaporator 4, and this mixed gas comes into contact with the catalyst, as it moves from the top to the bottom of the catalyst column, to promote the dehydrochlorination reaction. While the reaction gas that has passed through the bottom of the catalyst column passes through the collection tank filled with water, the reaction product, the unreacted starting material and hydrogen chloride are collected in the collection tank, and only the carrier gas is discharged outside the reaction system.

### [Purification of HFO-1447]

The high-purity HFO-1447 of the present invention can be obtained by distillation purification of a reaction liquid after carrying out step (b).

### [High-Purity HFO-1447]

The high-purity HFO-1447 obtained by the method of the present invention is characterized by having two isomers, an (E)-isomer and a (Z)-isomer, each of which has a purity of more than 99%, preferably 99.5% or more and more preferably 99.9% or more. Herein, unless other specified, “%” refers to a percentage by weight based on the 100% of the total weight.

### [Uses of High-Purity HFO-1447 of the Present Invention]

The high-purity HFO-1447 of the present invention is poorly soluble in an organic material, particularly very poorly soluble in an oil, but it is compatible with an organic solvent such as ethanol or 2-propanol. By taking advantage of such characteristics, it is useful for the following uses.

### (1) Uses as Solvent and Cleaning Agent

The high-purity HFO-1447 of the present invention can be mixed in any proportion with an organic solvent such as a ketone such as acetone or acetophenone; a nitrile such as acetonitrile or propionitrile: an ether such as diisopropyl ether, t-butyl methyl ether, tetrahydrofuran, diglyme or 1,4-dioxane; a sulfoxide such as dimethylsulfoxide or sulfolane: an amide such as dimethylformamide, dimethylacetamide or N-methylpyrrolidone; a hydrocarbon such as hexane, heptane, cyclohexane, benzene or toluene, and an alcohol such as methanol, ethanol or isopropanol. Therefore, it can be used as a mixed solvent in a wide range of uses. The boiling point of the high-purity HFO-1447 of the present invention is 52° C. for the (E)-isomer and 78° C. for the (Z)-isomer. Therefore, it can be easily volatilized and removed. Since the high-purity HFO-1447 of the present invention do not contain any allene compound as an impurity, it does not harden a resin when used, as validated in the Examples described below.

For example, more specific uses as a cleaning agent include the following three:
(i) particle cleaning:
   a cleaning liquid for removing fine particles from the surfaces of precision electronic devices:
(ii) co-solvent cleaning:
   a cleaning liquid for cleaning liquid crystal cells and liquid crystal panels, and for degreasing and drying workpieces; and
(iii) cleaning for dewatering and drying:
   a cleaning liquid for dewatering and drying lenses, sensor packages, semiconductor wafers, and other precision parts.

The high-purity HFO-1447 of the present invention has a low surface tension (E-isomer: 16.6 mN/m; Z-isomer: 18.9 mN/m) and a high specific gravity (E-isomer: 1.41 g/cm³: Z form: 1.44 g/cm³), and is easily dried, so that it is suitable for particle cleaning. In addition, the high-purity HFO-1447 of the present invention has a low surface tension, a low boiling point (E-isomer: 52° C.: Z-isomer: 78° C.), and incombustibility, so that it is also suitable for co-solvent cleaning. Furthermore, the high-purity HFO-1447 of the present invention has a low latent heat of evaporation (prevents dew condensation), incombustibility, a compatibility with a known cleaning agent (isopropanol) and high drying properties, so that it is also suitable for cleaning for dewatering and drying.

### (2) Use as Foaming Agent

The high-purity HFO-1447 of the present invention can be used to prepare a foaming composition of a thermosetting resin such as polyurethane and a thermoplastic resin such as polystyrene, polyethylene or polypropylene, by taking advantage of the miscibility thereof with an organic solvent.

### EXAMPLES

### [Example 1] Synthesis of 3-chloro-1,1,1,3,5,5,5-heptafluoropentane (HCFC-457) at Low Temperature in the Presence of SbCl₅ Catalyst

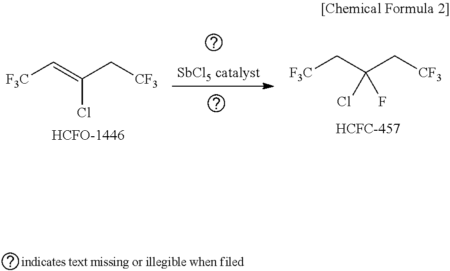 [SMILES: FC(F)(F)/C=C(\Cl)CC(F)(F)F ; FC(F)(F)CC(F)(Cl)CC(F)(F)F]

A 119.0 g portion (0.56 mol) of 3-chloro-1,1,1,5,5,5-hexafluoro-2-pentene (HCFO-1446) was placed in a 100 ml PFA (perfluoroalkoxy alkane) reactor equipped with a thermometer, a −20° C. chiller circulation condenser, and an inlet tube, and 12.3 g (0.61 mol, 1.1 eq.) of anhydrous HF (liquid) was introduced under cooling at −10° C. while stirring the reaction liquid. Then, 4.09 g (13.7 mmol, 2.5 mol %) of SbCl₅ was added, and the resultant was stirred at 0° C. for 48 hours. After stirring for 48 hours, 10 g of water was added under cooling at 0° C. to stop the reaction. The reaction liquid was transferred to a separatory funnel and separated to quantitatively obtain 130.29 g of an organic layer containing 3-chloro-1,1,1,3,5,5,5-heptafluoropentene with a GC purity of 99%.

### Example 2, Example 3, Example 4, Example 5 (Comparative), Example 6 (Comparative)

The reaction was carried out in the same manner as in Example 1, except that the reaction temperature, reaction time and HF equivalent were changed as shown in Table 1. The experimental results in Table 1 show that the target HCFC-457 can be obtained in a yield of 70% or more when selecting a reaction temperature as relatively low as 0 to 5° C., a reaction time as relatively long as 24 to 48 hours, and an HF equivalent of 1 to 2. In particular, when the reaction temperature is 0° C., the selectivity for HCFC-457 is high even if the HF equivalent is higher than the theoretical amount for HCFO-1446; however, when the reaction temperature is 5° C., HCFC-457 is further fluorinated to produce 1,1,1,3,3,5,5,5-octafluoropentane (HFC-458) if the HF equivalent is higher than the theoretical amount for HCFO-1446. It was also found that when the reaction temperature was −10° C. or less, the reaction did not proceed at all, and that when the reaction was carried out at an increased reaction temperature of 90° C. for a reaction time as short as 12 hours, the starting material HCFO-1446 was left and a large amount of HFC-458 was produced.

TABLE 1

| | | | | Post-reaction composition | | |
|---|---|---|---|---|---|---|
| | Reaction | | HF | HCFO- | HCFC- | HFC- |
| | conditions | | Equivalent | 1446 | 457 | 458 |
| No | ° C. | h | eq | % | % | % |
| 1 | 0 | 48 | 1.1 | 0 | 99 | <1 |
| 2 | 0 | 48 | 1.5 | 0 | 99 | <1 |
| 3 | 5 | 24 | 1.1 | 0 | 98 | 1 |
| 4 | 5 | 24 | 1.5 | 3 | 72 | 24 |
| 5 (Comparative) | −10 | 24 | 1.1 | 99 | 0 | 0 |
| 6 (Comparative) | 90 | 12 | 1.5 | 21 | 14 | 64 |

Note:
The post-reaction composition was determined by gas chromatographic (GC) area ratio.

### [Example 7] Synthesis of 1,1,1,3,5,5,5-heptafluoro-2-pentene by Dehydrochlorination of 3-chloro-1,1,1,3,5,5,5-heptafluoropentane with Activated Carbon Catalyst

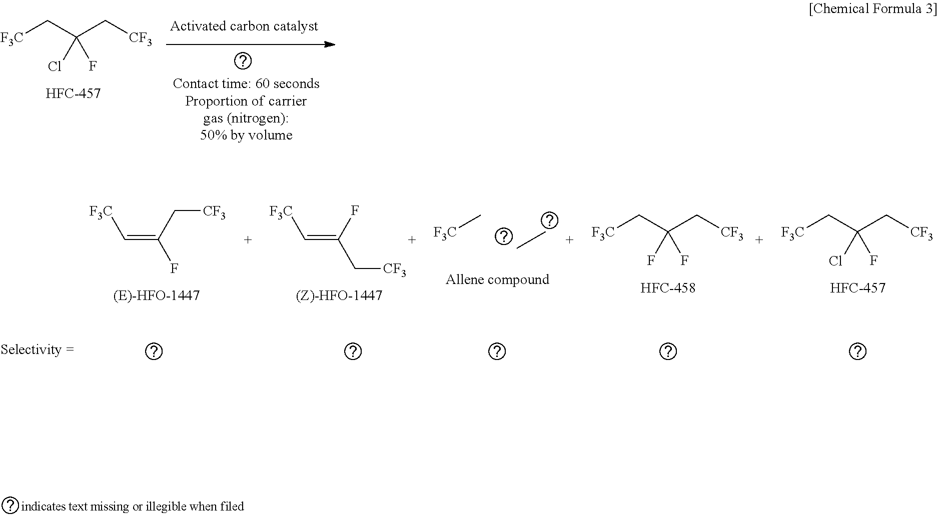

In the system shown in FIG. 1 including as a reactor a catalyst column (φ20.0×400 mm) packed with activated carbon pellets (Shirasagi C2X4/6-2), the catalyst column was supplied with 3-chloro-1,1,1,3,5,5,5-heptafluoropentane (HCFC-457) at 25.7 ml/h (2.80 mmol/min, 63 SCCM) under the condition of heating the catalyst column at 250° C. and an evaporator at 150° C. while allowing nitrogen to flow at 63 SCCM. The mixed gas of HCFC-457 and N2 was contacted with the activated carbon at a total flow rate of 125 SCCM for a contact time of 60 seconds, and a product was collected in a collection tank containing 300 g of ice water cooled to −2° C. After supplying HCFC-457 for 47 minutes, a pump was stopped. After white smoke was no longer seen in the collection tank, 23.22 g of an organic layer was recovered from the collection tank. Quantitative determination of the recovered organic phase by NMR showed that: any allene compound was not detected (N.D.) and HFC-458 was not detected (N.D.); the composition of the organic phase was 40.8% of (E)-1,1,1,3,5,5,5-heptafluoro-2-pentene, 57.2% of (Z)-1,1,1,3,5,5,5-heptafluoro-2-pentene and 2.0% of HCFC-457; and 1,1,1,3,5,5,5-heptafluoro-2-pentene (a mixture of an (E)-isomer and a (Z)-isomer) was obtained with a conversion rate of 98% and a selectivity of >99%.

### [Example 8] Distillation Purification of 1,1,1,3,5,5,5-heptafluoro-2-pentene Obtained in [Example 7]

A 150.0 g portion of a crude liquid with a composition of (E)-HFO-1447/(Z)-HFO-1447/an allene compound/HFC-458/HCFC-457=40.8%/57.2%/N.D./N.D./2.0% was subjected to distillation purification in a distillation column equipped with a Kiriyama Pac (packing: SUS net) having a theoretical plate number of 10 plates at a reflux ratio of 100/1. As a result, 55.0 g of (E)-HFO-1447 with a GC purity of >99% was obtained in a yield of 90% (the remaining 10% of the E isomer was obtained as a low purity fraction), and 80.7 g of (Z)-HFO-1447 with a GC purity of >99% was obtained in a yield of 94% (the remaining 6% of the Z isomer was obtained as a low purity fraction).

### [Example 9 (Comparative)] Synthesis of 1,1,1,3,5,5,5-heptafluoro-2-pentene by Dehydrofluorination Reaction of 1,1,1,3,3,5,5,5-octafluoropentane with AlF₃ Catalyst

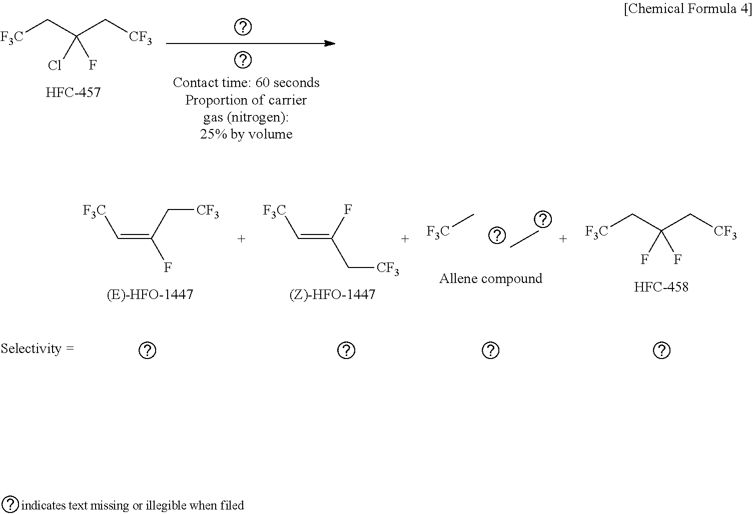

In the system shown in FIG. 1 including as a reactor a catalyst column packed with an AlF₃ catalyst, the catalyst column was supplied with 1,1,1,3,3,5,5,5-octafluoropentane (HFC-458) at 35.8 ml/h (4.2 mmol/min, 94 SCCM) under the condition of heating the catalyst column at 330° C. and an evaporator at 150° C. while allowing nitrogen to flow at 31 SCCM. The mixed gas of HFC-458 and N2 was contacted with the AlF₃ catalyst at a total flow rate of 125 SCCM for a contact time of 60 seconds, and a product was collected in a collection tank containing 200 g of water cooled to −2° C. After supplying HFC-458 for 60 minutes, a pump was stopped. After white smoke was no longer seen in the collection tank, 34.5 g of an organic phase was recovered from the collection tank. Quantitative determination of the recovered organic phase by NMR showed that: the composition of the organic phase was 4.3% of an allene compound, 37.6% of (E)-HFO-1447, 2.4% of HFC-458 and 55.6% of (Z)-HFO-1447; and 1,1,1,3,5,5,5-heptafluoro-2-pentene (a mixture of an (E)-isomer and a (Z)-isomer) was obtained with a conversion rate of 97.6% and a selectivity of 93.2%.

### Example 10 (Comparative), Example 11 (Comparative)

The reaction was carried out in the same manner as in Example 9, except that the heating temperature of a catalyst column was as shown in Table 2. Decreasing the reaction temperature resulted in a decrease in the amount of the allene compound generated as a by-product but an increase in the percentage of unreacted HFC-458.

TABLE 2

| | | Post-reaction composition | | | |
|---|---|---|---|---|---|
| | Reaction | (E)-HFO- | (Z)-HFO- | Allene | HFC- |
| | conditions | 1447 | 1447 | compound | 458 |
| No | ° C. | % | % | % | % |
| 9 | 330 | 37.6 | 55.6 | 4.3 | 2.4 |
| (Comparative) | | | | | |
| 10 | 250 | 33.9 | 46.6 | 0.3 | 19.2 |
| (Comparative) | | | | | |
| 11 | 300 | 37.1 | 55.3 | 1.4 | 6.3 |
| (Comparative) | | | | | |

Note:
The post-reaction composition was determined by gas chromatographic (GC) area ratio.

### [Example 12 (Comparative)] Distillation Purification of 1,1,1,3,5,5,5-heptafluoro-2-pentene Obtained by the Method of [Example 11 (Comparative)]

A 152.0 g portion of a crude liquid with a composition of (E)-HFO-1447/(Z)-HFO-1447/an allene compound/HFC-458=37.1%/55.3%/1.4%/6.3% was subjected to distillation purification in a distillation column equipped with a Kiriyama Pac (packing: SUS net) having a theoretical plate number of 10 plates at a reflux ratio of 200/1. As a result, 26.4 g of (E)-HFO-1447 with a GC purity of >99% was obtained in a yield of 45% (the remaining 55% of the E isomer was obtained as a low purity fraction), and 39.0 g of (Z)-HFO-1447 with a GC purity of >99% was obtained in a yield of 51% (the remaining 49% of the Z isomer was obtained as a low purity fraction). Each of the isomers was obtained with a high purity, but it was found that when eliminating contamination with the impurities, the amount of the low purity fraction increased and the yield of the target product decreased.

### [Example 13 (Comparative)] Synthesis of 1,1,1,3,5,5,5-heptafluoro-2-pentene by Dehydrofluorination Reaction of 1,1,1,3,3,5,5,5-octafluoropentane with Activated Carbon Catalyst

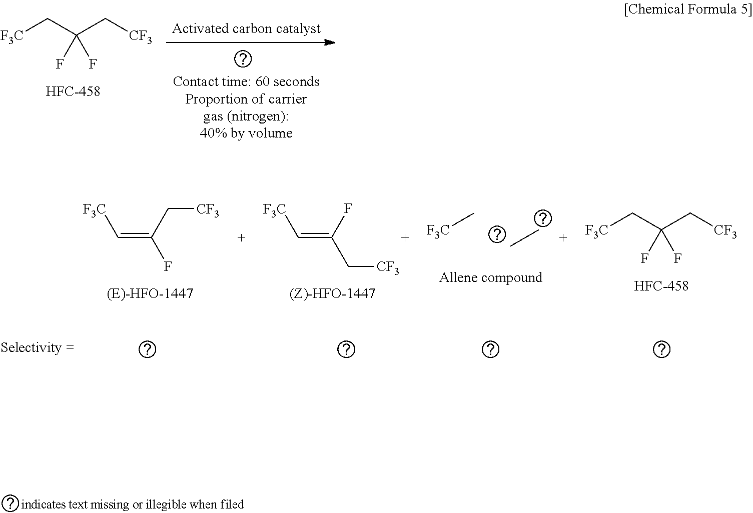

In the system shown in FIG. 1 including as a reactor a catalyst column (φ20.0×400 mm) packed with activated carbon pellets (Shirasagi C2X4/6-2), the catalyst column was supplied with 1,1,1,3,3,5,5,5-octafluoropentane (HFC-458) at 23.9 ml/h (2.8 mmol/min, 63 SCCM) under the condition of heating the catalyst column at 250° C. and an evaporator at 150° C. while allowing nitrogen to flow at 63 SCCM. The mixed gas of HFC-458 and N2 was contacted with the activated carbon at a total flow rate of 125 SCCM for a contact time of 60 seconds, and a product was collected in a collection tank containing 200 g of ice water cooled to −2° C. After supplying HFC-458 for 51 minutes, a pump was stopped. After white smoke was no longer seen in the collection tank, 26.93 g of an organic phase was recovered from the collection tank. Quantitative determination of the recovered organic phase by NMR showed that: any allene compound was not detected (N.D.); the composition of the organic phase was 76.3% of HFC-458, 7.8% of (E)-HFO-1447 and 15.9% of (Z)-HFO-1447; and HFO-1447 (a mixture of an (E)-isomer and a (Z)-isomer) was obtained with a conversion rate of 23.7% and a selectivity of >99%. It was found that when HFC-458 was subjected to a dehydrofluorination reaction to produce HFO-1447, the elimination reaction was unlikely to occur and a large amount of the starting material HFC-458 was left in the product.

### [Example 14] Resin Material Test for (E)-HFO-1447 with GC Purity of >99%

A 10.0 mm×10.0 mm×1.0 mm test piece, the dimensions and weight of which had been previously measured, was placed in a 20 ml glass bottle. 5 g of (E)-HFO-1447 with a GC purity of >99% was added thereto, and the glass bottle was then sealed. The bottle was stored in a thermostat bath heated to 52° C. for 3 hours. After a predetermined time, the glass bottle was removed from the thermostatic bath and allowed to cool at room temperature. Thereafter, the glass bottle was opened, and the state of the solution and changes of the test piece were observed and the dimensions and weight of the test piece were measured. The results are shown in Table 3. The criteria for evaluation are as follows:
Good (circle): No corrosion or damage, no weight change, no volume change of the resin
Fair (triangle): No corrosion or damage of the resin, but weight change and volume change of the resin
Poor (cross mark): Corrosion or damage of the resin

### [Example 15 (Comparative)] Resin Material Test for (E)-HFO-1447 Containing 3% by Weight of Allene Compound

The evaluation was carried out in the same manner as in [Example 14]. The results are shown in Table 3. The reason why the concentration of the allene compound was 3% by weight is that, from the experimental results of Example 11 (Comparative), it was assumed that the (E)-HFO-1447 obtained by the production method of PTL 1 contained the allene compound at a ratio of the (E)-HFO-1447: the allene compound of about 97% by weight: 3% by weight.

### [Example 16] Resin Material Test for (Z)-HFO-1447 with GC Purity of >99%

The evaluation was carried out in the same manner as in [Example 14], except that the heating temperature was 77° C. The results are shown in Table 4.

### [Example 17 (Comparative)] Resin Material Test for (Z)-HFO-1447 Containing 10% by Weight of HFC-458

The evaluation was carried out in the same manner as in [Example 14], except that the heating temperature was 77° C. The results are shown in Table 4. The reason why the concentration of HFC-458 was 10% by weight is that, from the experimental results of Example 11 (Comparative), it was assumed that the (Z)-HFO-1447 obtained by the production method of PTL 1 contained HFC-458 at a ratio of the (Z)-HFO-1447: HFC-458 of about 90% by weight: 10% by weight.

### [Example 18] Elastomer Material Test for (E)-HFO-1447 with GC Purity of >99%

The evaluation was carried out in the same manner as in [Example 14]. The results are shown in Table 5.

### [Example 19 (Comparative)] Elastomer Material Test for (E)-HFO-1447 Containing 3% by Weight of Allene Compound

The evaluation was carried out in the same manner as in [Example 14]. The results are shown in Table 5. The reason why the concentration of the allene compound was 3% by weight is that, from the experimental results of Example 11 (Comparative), it was assumed that the (E)-HFO-1447 obtained by the production method of PTL 1 contained the allene compound at a ratio of the (E)-HFO-1447: the allene compound of about 97% by weight: 3% by weight.

TABLE 3
Resistance test of resins to (E)-HFO-1447
and (E)-HFO-1447 containing allene compound

| | (E)-HFO-1447 | (E)-HFO-1447 + |
|---|---|---|
| Test specimen | alone | Allene compound |
| PTFE | Good | Fair |
| | | (Weight + 1.0%) |
| Polyethylene | Good | Fair |
| (low density) | | (Weight + 1.1%) |
| Soft PVC | Good | Poor |
| (polyvinyl chloride) | | (Hardened) |
| Nylon 66 | Good | Fair |
| | | (Weight − 0.9%) |
| Polypropylene | Good | Fair |
| | | (Weight + 0.9%) |

The experimental results in Table 3 show that the production method of the present invention can efficiently produce a high-purity (E)-HFO-1447 that causes no corrosion or damage or no weight change of the resin. The conventional production method can provide a high-purity (E)-HFO-1447 when precision distillation is carried out, but it will be accompanied by a significant decrease in yield due to the removal of a low purity fraction.

TABLE 4
Resistance test of resins to (Z)-HFO-1447
and (Z)-HFO-1447 containing HFC-458

| | (Z)-HFO-1447 | (Z)-HFO-1447 + |
|---|---|---|
| Test specimen | alone | HFC-458 |
| PTFE | Fair | Fair |
| | (Weight + 0.8%) | (Weight + 2.0%) |
| Polyethylene | Fair | Poor |
| (high density) | (Weight + 0.6%) | (Component was extracted) |
| Polyethylene | Fair | Poor |
| (low density) | (Weight + 1.3%) | (Whitened) |
| Nylon 66 | Fair | Fair |
| | (Weight − 1.4%) | (Weight − 2.2%) |
| Nylon 6 | Fair | Fair |
| | (Weight − 1.9%) | (Weight − 3.1%) |
| | | (Volume − 2.8%) |
| PVDF | Fair | Fair |
| | (Weight + 1.0%) | (Weight + 7.3%) |
| | | (Volume + 5.3%) |
| Polypropylene | Fair | Poor |
| | (Weight + 0.9%) | (Whitened) |
| Polycarbonate | Good | Fair |
| | | (Weight + 1.8%) |
| ABS | Fair | Poor |
| | (Weight + 2.5%) | (Partially dissolved) |
| Polystyrene | Good | Poor |
| | | (Curved) |

The experimental results in Table 4 show that the production method of the present invention can efficiently produce a high-purity (Z)-HFO-1447 that causes no corrosion or damage of the resin or causes no or little weight change of the resin. The conventional production method can provide a high-purity (Z)-HFO-1447 when precision distillation is carried out, but it will be accompanied by a significant decrease in yield due to the removal of a low purity fraction.

TABLE 5
Resistance test of elastomers to (E)-HFO-1447
and (E)-HFO-1447 containing allene compound

| | (E)-HFO-1447 | (E)-HFO-1447 + |
|---|---|---|
| Test specimen | alone | Allene compound |
| Chloroprene rubber | Good | Poor |
| | | (Filler deposition) |
| Butyl rubber | Good | Poor |
| | | (Solvent colored yellow) |
| | | (Whitened) |
| EPDM | Good | Poor |
| | | (Filler deposition) |
| Silicone rubber | Fair | Fair |
| | (Weight + 13%) | (Weight + 14.0%) |
| | (Volume + 3%) | (Volume + 7.8%) |
| NBR | Fair | Poor |
| | (Weight + 1.3%) | (Filler deposition) |
| Urethane rubber | Fair | Fair |
| | (Weight + 14%) | (Weight + 20.5%) |
| | (Volume + 2%) | (Volume + 19.0%) |

The experimental results in Table 5 show that the production method of the present invention can efficiently produce a high-purity (E)-HFO-1447 that causes no corrosion, weight change or volume change of the elastomer or causes little weight change or volume change of the elastomer. The conventional production method can provide a high-purity (E)-HFO-1447 when precision distillation is carried out, but it will be accompanied by a significant decrease in yield due to the removal of a low purity fraction.

### [Example 20] Compatibility Test of (E)-HFO-1447 and (Z)-HFO-1447 with GC Purity >99% with Alcohol Solvent

(E)-HFO-1447 or (Z)-HFO-1447 with a GC purity of >99% and an alcohol solvent were placed in appropriate amounts in 20 ml glass bottles, and it was visually observed whether phase separation occurred. The results are shown in Table 6. The criteria for evaluation are as follows:
Good (circle): No phase separation, and compatible
Fair (triangle): Phase separation, but compatible by stirring
Poor (cross mark): Phase separation

### [Example 21 (Comparative)] Compatibility Test of Commercially Available fluorobutene and perfluoroheptene with Alcohol Solvent

The evaluation was carried out in the same manner as in [Example 20]. The results are shown in Table 6.

TABLE 6

| Object | | | | |
|---|---|---|---|---|
| to be | | | Commercially | Commercially |
| evaluated | | | available | available |
| Material | (E)-HFO- | (Z)-HFO- | cleaning agent | cleaning agent |
| name | 1447 | 1447 | Fluorobutene | Perfluoroheptene |
| Ethanol | Good | Good | Good | Poor |
| | (Compat- | (Compat- | (Compatible) | (Phase separation) |
| | ible) | ible) | | |
| 2- | Good | Good | Fair | Poor |
| Propanol | (Compat- | (Compat- | (Compatible by | (Phase separation) |
| | ible)) | ible)) | stirring) | |

The experimental results in Table 6 show that the production method of the present invention can efficiently produce a high-purity (E)- or (Z)-HFO-1447 that is compatible with alcohol solvents, particularly isopropanol (2-propanol). However, both of commercially available fluorobutene and perfluoroheptene, which are fluorine-based cleaning agents, have problems with compatibility with isopropanol. In particular, since isopropanol is used as a cleaning agent for semiconductor wafers, the high-purity HFO-1447 of the present invention may also be useful for a cleaning agent when mixed with other cleaning solvents.

From the above results, it was found that each of (E)- and (Z)-HFO-1447 to be produced by the method of the present invention is unlikely to deteriorate the resin and is suitable for use as a cleaning agent, since it is produced such that it does not contain by-products that are otherwise generated in the reaction system and are difficult to remove, and then purified.

1. A method for producing 1,1,1,3,5,5,5-heptafluoro-2-pentene, comprising:
(a) reacting 3-chloro-hexafluoro-2-pentene with hydrogen fluoride at a temperature of more than −10° C. and 20° C. or less in the presence of a metal halide catalyst to produce 3-chloro-1,1,1,3,5,5,5-heptafluoropentane; and
(b) subjecting the 3-chloro-1,1,1,3,5,5,5-heptafluoropentane obtained in (a) to a dehydrochlorination reaction in the presence of an activated carbon catalyst to produce 1,1,1,3,5,5,5-heptafluoro-2-pentene.

2. The method according to claim 1, wherein the metal halide catalyst is selected from an antimony halide catalyst, a tin halide catalyst, a titanium halide catalyst, a niobium halide catalyst, a tantalum halide catalyst or a combination thereof.

3. The method according to claim 1, wherein the metal halide catalyst is selected from antimony trichloride, antimony pentachloride, antimony trifluoride, antimony pentafluoride, tin tetrachloride, titanium tetrachloride, niobium pentafluoride, tantalum pentafluoride or a combination thereof.

4. The method according to claim 1, wherein hydrogen fluoride is used at a molar equivalent ratio of 1 to 1.5 of hydrogen fluoride to 3-chloro-hexafluoro-2-pentene.

5. The method according to claim 1, wherein the amount of the metal halide catalyst is 2 to 3 mol % based on the amount of 3-chloro-hexafluoro-2-pentene.

6. The method according to claim 1, wherein the reacting of step (a) is carried out at a temperature of −5° C. to 10° C.

7. The method according to claim 1, wherein the metal halide catalyst is selected from antimony trichloride, antimony pentachloride, antimony trifluoride, antimony pentafluoride, tin tetrachloride, titanium tetrachloride, niobium pentafluoride, tantalum pentafluoride or a combination thereof, hydrogen fluoride is used at a molar equivalent ratio of 1 to 1.5 of hydrogen fluoride to 3-chloro-hexafluoro-2-pentene; the amount of the metal halide catalyst is 2 to 3 mol % based on the amount of 3-chloro-hexafluoro-2-pentene; and the reacting in step (a) is carried out at a temperature of −5° C. to 10° C.

8. A high-purity 1,1,1,3,5,5,5-heptafluoro-2-pentene obtained by the method according to claim 1.

9. The high-purity 1,1,1,3,5,5,5-heptafluoro-2-pentene according to claim 8, having a purity of more than 99%.
